(19) United States
(12) Patent Application Publication
(10) Pub. No.: US 2017/0128063 A1
(43) Pub. Date: May 11, 2017

(54) TISSUE CINCHING SUTURE SYSTEM THAT IS SLIDELESS AGAINST THE TISSUE DURING CINCHING

(71) Applicant: Jeffrey David Jackson, Salt Lake City, UT (US)

(72) Inventor: Jeffrey David Jackson, Salt Lake City, UT (US)

(21) Appl. No.: 15/348,765

(22) Filed: Nov. 10, 2016

Related U.S. Application Data
(60) Provisional application No. 62/253,457, filed on Nov. 10, 2015.

(51) Int. Cl. A61B 17/04; A61B 17/06

(52) U.S. Cl. CPC A61B 17/0485; A61B 17/0401; A61B 17/06066; A61B 2017/0404; A61B 2017/0409; A61B 2017/044

(57) ABSTRACT

A suture implant device includes: a splice; a fixed loop having a first fixed end at a first end of the splice and a second fixed end at the first end of the splice; an adjustable loop having a fixed end at the second end of the splice and an adjustable end at the second end of the splice, the adjustable end passes through the at least a portion of the splice so that a tensioning strand extending from the adjustable loop protrudes from the splice; a needle on the fixed loop; and a member on the adjustable loop. A method of cinching a first tissue to a second tissue includes: attaching the suture implant to a first tissue; attaching the suture implant to a second tissue; and cinching the first tissue to the second tissue without the suture implant sliding against or through the first tissue.

### CROSS-REFERENCE

This patent application claims priority to U.S. Provisional Application No. 62/253,457 filed Nov. 10, 2015, which provisional is incorporated herein by specific reference in its entirety.

### BACKGROUND

It has been found that traditional sutures and use thereof to cinch two different tissues together can injure one or both tissues and complicate the surgical procedure. The suture can unfavorably include the cord passing through one of the tissues in a manner where the cord slides against the tissue, such as sliding against a wall of a hole in the tissue, and acts as a saw to slice through the tissue or otherwise tear the tissue. The sliding action of the cord can abrade the tissue and wear or otherwise tear down the structure of the tissue, which can cause the hole to increase in size and take on an injury from the cut of the sliding cord. In some instances, pulling tension onto a tissue that has the suture cord sliding against the tissue can tear the tissue and pull the suture cord from the injured tissue.

### SUMMARY

In one embodiment, a suture implant device can include: a fixed loop splice; a fixed loop having a first fixed end at a first end of the fixed loop splice and the fixed loop having a second fixed end at the first end of the fixed loop splice; an adjustable loop splice having a first end and a second end that is adjacent to, spaced from, connected with or integrated with a second end of the fixed loop splice; an adjustable loop having a fixed end at the first end of the adjustable loop splice and the adjustable loop having an adjustable end at the first end of the adjustable loop splice, wherein the fixed end forms the adjustable loop splice and the adjustable end passes through the adjustable loop splice so that a tensioning strand extending from the adjustable loop protrudes from the second end of the adjustable loop splice; a needle on the fixed loop; and a member on the adjustable loop. In one aspect, the fixed end includes a cord extending away from the adjustable loop splice and then back into and through the adjustable loop splice to extend for form the tensioning strand. In one aspect, the first fixed end and second fixed end include a cord extending away from the fixed loop splice and connected back to it so as to form the fixed loop. In one aspect, a cord connected to one of the first fixed end or second fixed end of the fixed loop protrudes from the fixed loop splice and has an anchor member on its end to inhibit being drawn into the fixed loop splice, thereby fixing the fixed loop. In one aspect, the needle is slidably on the fixed loop. In one aspect, the member is slidably on the adjustable loop. In one aspect, the member is a button, suture, screw, plate, bone anchor, or any implant of any type, such as any orthopedic device. In one aspect, the member comprises a body, button, anchor, or second implant having one hole or at least two spaced apart holes each being dimensioned to receive the cord of the adjustable loop therethrough. In one aspect, the adjustable loop splice has the second end that is at least partially integrated with a second end of the fixed loop splice. In one aspect, the tensioning strand extending from the adjustable end of the adjustable loop extends into, through and out from any portions of the adjustable loop splice. In one aspect, the tensioning strand of the adjustable loop has an anchor member that inhibits a free end of the tensioning strand from being pulled through the adjustable loop splice.

In one embodiment, a suture implant device includes: a braided splice having a first end and a second end; a first strand braided from a first end of the braided splice and extending therefrom; a second strand braided from the first end of the braided splice and extending therefrom, wherein the first strand and second strand connect to form a fixed loop; and a third strand braided from the second end of the braided splice and extending therefrom and looped back thereto and passing into a lumen in the second end of the braided splice to form an adjustable loop and further extending through the lumen and from the braided splice. In one aspect, an anchor member is on the braided splice that inhibits the braided splice from being pulled through a tendon or other tissue. In one aspect, an anchor member is on the third strand opposite of the adjustable loop that inhibits the end of the third strand from being pulled through the braided splice. In one aspect, an anchor member is on the adjustable loop. In one aspect, a needle is on the fixed loop. In one aspect, the braided splice, first strand, and third strand are formed from a single braided cord. In one aspect, the braided splice, first strand, second strand and third strand are formed from a single braided cord, where the first strand and second strand branch from the braided splice.

In one embodiment, a method of cinching a first tissue to a second tissue with the suture implant device of one of the claims, the method comprising: obtaining a suture implant; attaching the suture implant to a first tissue; attaching the suture implant to a second tissue; and cinching the first tissue to the second tissue with the suture implant without the suture implant sliding against or through the first tissue. In one aspect, the method can include: stitching the fixed loop to the first tissue; attaching the member on the adjustable loop to the second tissue; and cinching the first tissue to the second tissue by pulling on the tensioning strand to cinch close the adjustable loop. In one aspect, the method can include releasing the tensioning strand so that the splice traps the cord connecting the adjustable end of the adjustable loop with the tensioning strand within the splice so that inner walls of a lumen of the splice apply pressure to the cord. In one aspect, the method can include fixing the tensioning strand so that the adjustable loop is fixed and no longer adjustable.

In one embodiment, the method can include providing a suture implant device comprising: at least one splice; a fixed loop having a first fixed end at a first end of the at least one splice and the fixed loop having a second fixed end at the first end of the at least one splice; an adjustable loop having a fixed end at a second end of the splice and the adjustable loop having an adjustable end at the second end of the splice, wherein the adjustable end passes through the at least a portion of the at least one splice so that a tensioning strand extending from the adjustable loop protrudes from the at least one splice; a needle on the fixed loop; and a member on the adjustable loop.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A shows an embodiment of a suture implant.

FIG. 1B shows a portion of the suture implant being a splice with an outer sheath portion and inner cord portion.

FIG. 2 shows a braided splice having a braided sheath that splits at least at one end into two separate cord members.

FIG. 3 shows an embodiment of a suture implant having the braided splice of FIG. 2.

FIG. 4 shows an embodiment of a method of cinching a first tissue to a second tissue with a suture implant.

FIG. 4A-4E show steps of a method of cinching a first tissue to a second tissue with a suture implant.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention is a suture implant that can be attached to a first tissue and cinch the first tissue to a second tissue without the cinching causing the suture implant or anything else to slide through the first tissue during the cinching. As a result, the suture implant provides a slideless suture that does not slide against the first tissue. The lack of sliding against the first tissue helps inhibit damage to the first tissue that may occur if the suture slides against the first tissue. Accordingly, the implant can cinch the first tissue and second tissue together without damaging the first tissue.

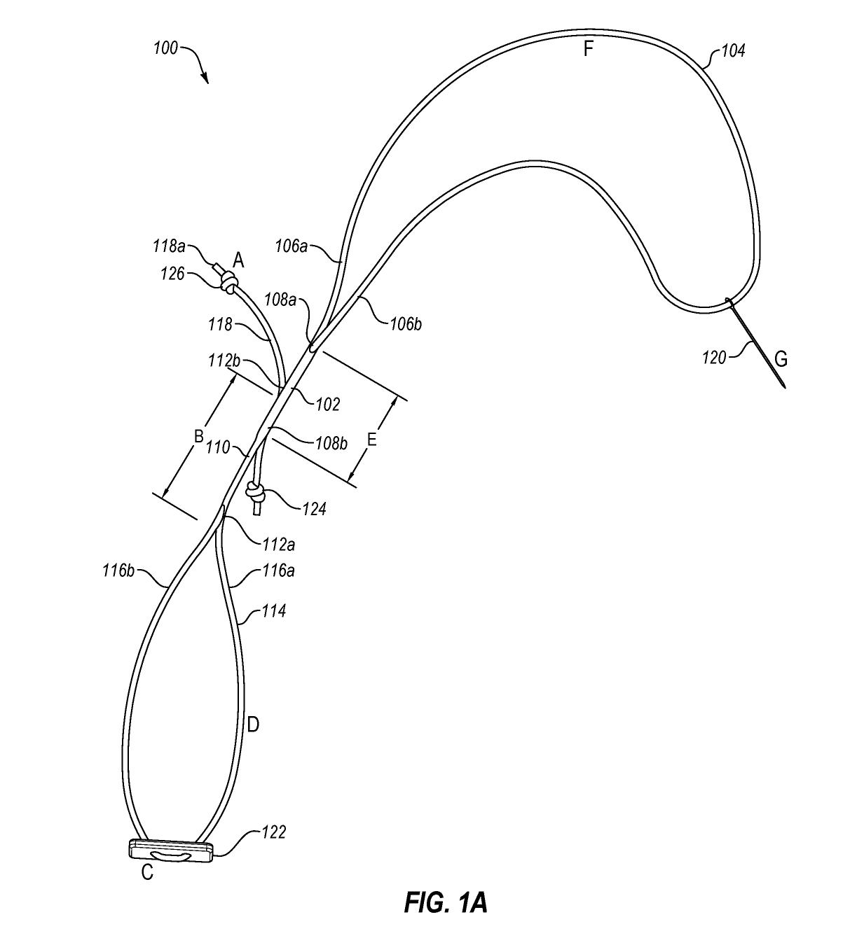

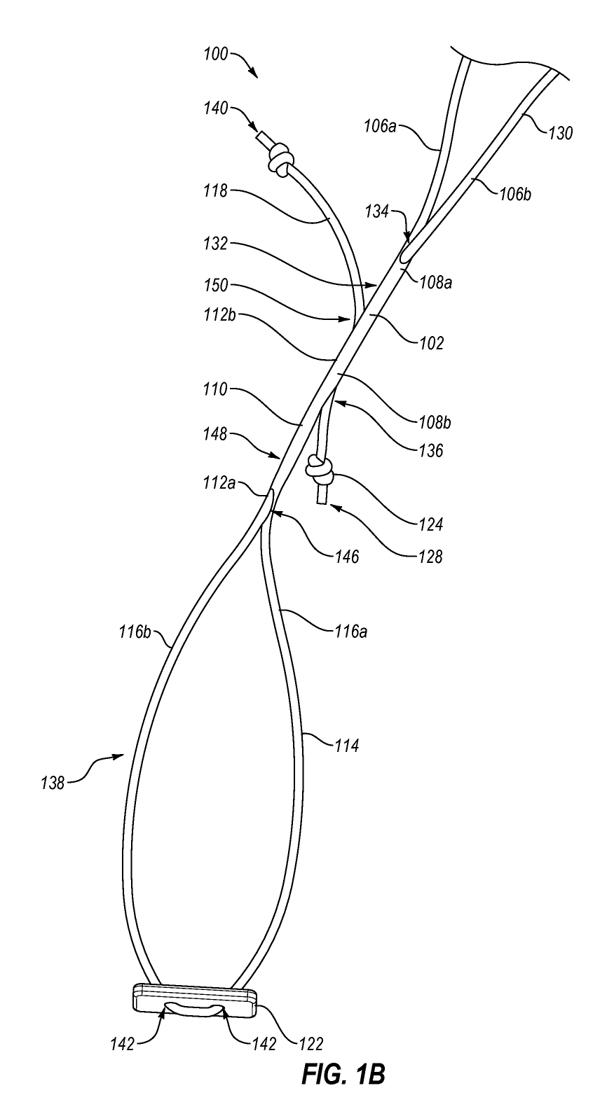

FIGS. 1A-1B illustrates an embodiment of a suture implant device 100. The suture implant device 100 can include a fixed loop splice 102 having a dimension E from a first end 108a to a second end 108b. The suture implant device 100 can include a fixed loop 104 with a dimension F and having a first fixed end 106a at a first end 108a of the fixed loop splice 102 and the fixed loop 104 having a second fixed end 106b also at the first end 108a of the fixed loop splice 102. One of the first fixed end 106a or second fixed end 106b can form the fixed loop splice 102 with the suture of the other fixed end being inserted within the suture so that the external suture strand is the sheath having the internal suture strand passing therethrough. The suture implant device 100 also includes an adjustable loop splice 110 having a dimension B and having a first end 112a and a second end 112b that are adjacent to, spaced from, connected with or integrated with a second end 108b of the fixed loop splice 102. That is, the fixed loop splice 102 and adjustable loop splice 110 can be attached to each other or separate, as well as overlap, and even can have the sheath of both the fixed loop splice 102 and adjustable loop splice 110 formed from a continuous cord portion of the suture. The suture implant device 100 includes an adjustable loop 114 having an adjustable dimension D having a fixed end 116a at the first end 112a of the adjustable loop splice 110 and the adjustable loop 114 having an adjustable end 116b also at the first end 112a of the adjustable loop splice 110. The fixed end 116a forms the sheath portion (e.g., outer portion) of the adjustable loop splice 110 and the adjustable end 116b passes through the adjustable loop splice 110 so that a tensioning strand 118 having an adjustable dimension A of the adjustable loop splice 110 that protrudes from the second end 112b of the adjustable loop splice 110. This allows the cord portion of the adjustable end 116b to be within the sheath of the fixed end 116a. The suture implant device 100 can include a needle 120 with a suitable gauge G on the fixed loop 104, where the fixed loop 104 passes through an eyelet of the needle 120. The suture implant device 100 can include a member 122 having a configuration C on the adjustable loop 114 such that the member 122 slides over the adjustable loop 114.

In one aspect, one end of the first fixed end 106a or second fixed end 106b of the fixed loop 104 terminates in the fixed loop splice 102. In one aspect, one end of the first fixed end 106a or second fixed end 106b of the fixed loop 104 protrudes from the fixed loop splice 102. In one aspect, one end of the first fixed end 106a or second fixed end 106b of the fixed loop 104 protrudes from the fixed loop splice 102 at a location and has an anchor member 124 having a configuration H on its end to inhibit being drawn into the fixed loop splice 102. The point at the fixed loop splice 102 from which the fixed end having the anchor member 124 protrudes therefrom is considered to be the second end 108b.

In one aspect, the needle 120 is slidably on the fixed loop 104, such as by having an eyelet that receives the fixed loop 104 therethrough. In one aspect, the member 122 is slidably on the adjustable loop 114, such as by having at least one eyelet or aperture (e.g., two eyelets or apertures) that receives the adjustable loop 114 therethrough. In one aspect, the member 122 is a button, suture, screw, plate, bone anchor, or any implant of any type, such as any orthopedic device. Often, the member 122 is configured to attach to a tissue, such as a hard tissue (e.g., bone), and thereby can be configured as a bone anchor or screw. In one aspect, the member 122 is a body, button, anchor, or second implant having at least two spaced apart holes each being dimensioned to receive the cord of the adjustable loop 114 therethrough so that the adjustable loop 114 passes through both spaced apart holes.

In one aspect, the adjustable loop splice 110 can have the second end 112b that is at least partially integrated with a second end 108b of the fixed loop splice 102.

In one aspect, the tensioning strand 118 of the adjustable loop 114 extends from any portion of the adjustable loop splice 110. The tensioning strand 118 of the adjustable loop 114 may optionally have an anchor member 126 (e.g., knot) that inhibits a free end 118a of the tensioning strand 118 from being pulled through the adjustable loop splice 110.

The suture implant device 100 can include the adjustable loop 114 that has the tensioning strand 118 attached thereto so that pulling the tensioning strand 118 cinches the adjustable loop 114 closed. This allows a practitioner to shorten the adjustable loop 114 of the implant 100. The adjustable loop 114 has a member 122 on it, where the member 122 may be a bone anchor that fastens into the bone. The implant 100 includes a fixed loop 104 that has a needle 120 on it that allows the practitioner to be able to whipstitch a tendon (e.g., in a fashion that is more like a baseball stitch) or other multi-stitch that secures the fixed loop splice 102 and/or cords of the fixed loop 104 to be fixed to the tendon (e.g., first tissue). The needle 120 on the fixed loop 104 allows the practitioner to lock the suture to the first tissue in a way that is useful and allows the practitioner to be able to whipstitch the implant 100 on the first tissue (e.g., tendon). The fixed loop 104 with the needle 120 on it is connected to the fixed loop splice 102, and the fixed loop splice 102 is connected to or adjacent to the adjustable loop splice 110. The fixed loop 104 extending from the first end 108a of the fixed loop splice 102 and having the needle 120 on the fixed loop 104 allows for a unique whipstitching for securing to the first tissue such that when used and implanted, no cord of the implant 100 slides against or through a hole in the first tissue.

The anchor member 124 (e.g., knot) on one of the ends of the fixed loop 104 can be used to stop the implant 100 from passing through the tendon. If the needle 120 is pushed through the tendon the anchor member 124 will stop the implant 100 from going all the way through the tendon. The anchor member 124 will stop the implant 100 at a point (e.g., location where the second fixed end 106b exits the fixed loop splice 102) for securing the fixed loop 104 to the tendon. As such, the anchor member 124 functions as an optional stop mechanism. This can result in the fixed loop splice 102 being inserted through a hole in the tendon.

As with many suture implant devices, the implant 100 can be prepared from a single cord. However, multiple cords with or without dedicated sheaths for the splice sheaths may be used to prepare the implant. Here, the fabrication of the implant from a single cord is explained in an example, although other techniques may be used to arrive at the same implant. FIG. 1B shows the features for such formation. The cord 130 is held at a region 132 adjacent to the first fixed end 106a and the cord end 128 is passed through an eyelet of the needle 120 and then a point 134 (shown by the dashed lines) at the first fixed end 106a is opened so that the cord end 128 is passed therein so that the cord 130 at the region 132 forms a lumen around the cord 130 to the cord end 128, and the cord end 128 is slid within the lumen of the cord 130 until reaching a point 136 (shown by dashed lines) at which the cord end 128 is pulled out from the lumen of the cord 130. The region where the cord 130 is within the cord 130 between point 134 and point 136 is the fixed loop splice 102, with the outer cord portion being the sheath of the inner cord portion. The anchor member 124 is then formed or attached to the cord 130, such as at the cord end 128 and then the anchor member 124 is pulled against the cord 130 at point 136 so that it no longer can be pulled, which forms the fixed loop 104. The cord 130 of the fixed loop 104 that is inserted into point 134 then is the second fixed end 106b. A cord region 138 that extends away from the fixed loop splice 102 opposite of the fixed loop 104 has a second cord end 140 that is passed through eyelets or holes 142 in the member 122 (e.g., into hole 142 from one side of the member 122 around the other side of the member 122 and then through hole 144 to exit from the one side of the member 122). The cord 130 at point 146 (shown by dashed line) is then opened and second cord end 140 is then inserted therein so that the cord 130 at region 148 forms a lumen with the second cord end sliding therethrough until reaching point 150 (shown by dashed line) at which the second cord end 140 is pulled out from the lumen of the cord 130. The region 148 between point 146 and 150 is the adjustable loop splice 110, with the outer cord portion being the sheath of the inner cord portion. Also, the insertion of the second cord end 140 into point 146 forms the adjustable loop 114 having the member 122 slidably thereon. The second cord end 140 is pulled further from point 150 to form the tensing strand 118. This allows the tensing strand 118 to be pulled from point 150

Of course, the location of points 134, 136, 146, and 150 can vary along the cord 130. Points 136 and 150 may be close, at a same location, or separated so that the adjustable loop splice 110 and the fixed loop splice 102 overlap or are separated. However, there needs to be some dimension B for the adjustable loop splice 110 and some dimension E for the fixed loop splice 102.

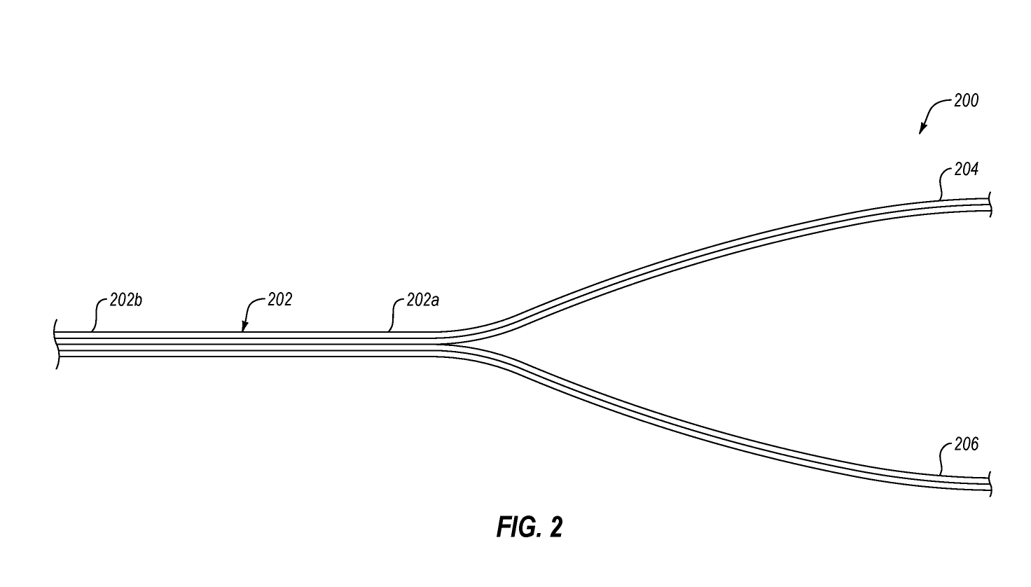

FIG. 2 illustrates an integrated splice 200. The integrated splice 200 can be used for the fixed loop splice, the adjustable loop splice, and for a combination of the fixed loop splice and adjustable loop splice. The integrated splice 200 can include a single strand braided splice 202 having a first end 202a and a second end 202b. A first strand 204 can be connected (e.g., braided) from the first end 202a of the braided splice 202 and extending therefrom. A second strand 206 can be connected (e.g., braided) from the first end 202a of the braided splice 202 and extending therefrom. In one aspect, the braided splice 202 is a fixed loop splice. In one aspect, the first strand 204 and second strand 206 connect to form a fixed loop. In one aspect, the braided splice 202 is a splice for a fixed loop and splice for an adjustable loop. In one aspect, an adjustable loop (not shown) extends from the second end 202b of the braided splice 202. In one aspect, an anchor member (not shown) is located on the braided splice 202 that inhibits the braided splice 202 from being pulled through a tendon or other tissue. As such, the subject matter of FIG. 2 can be applied to the splices of FIGS. 1A-1B.

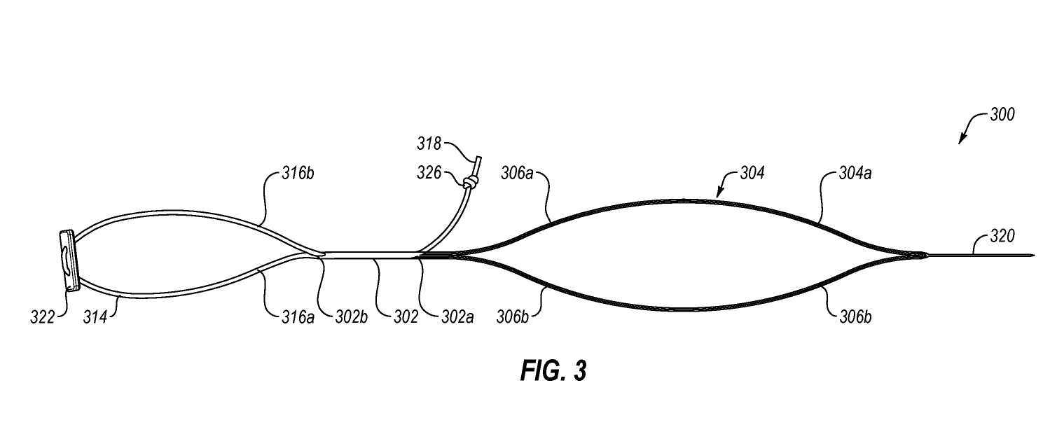

FIG. 3 illustrates an embodiment of a suture implant device 300 similar to the implant 100 of FIGS. 1A-1B, except that the braided splice 202 of FIG. 2 is included for both the fixed loop splice 102 and adjustable loop splice 110. The suture implant device 300 can include a splice 302 (e.g., braided splice of FIG. 2). The suture implant device 300 can include a fixed loop 304 having a first fixed end 306a (e.g., from first strand 204) of first strand 304a at a first end 302a of the splice 302 and the fixed loop 304 having a second fixed end 306b (e.g., from second strand 206) of second strand 304b at the first end 302a of the splice 302. The suture of the splice 302 splits as shown in FIG. 2 to form the first strand 304a and second strand 304b. The first strand 304a and second strand 304b are then connected, such as both being connected to the needle 320 to form the fixed loop 304. The suture implant device 300 also has the other end of the splice 302 (opposite of the fixed loop 304) connected to an adjustable loop 314 having a fixed end 316a at the second end 302b of the splice 302 and the adjustable loop 314 having an adjustable end 316b at the second end 302b of the splice 302. The fixed end 316a can form the splice 302 or part thereof or attach thereto, and the adjustable end 316b passes through the splice 302 so that a tensioning strand 318 of the adjustable loop 314 protrudes from the second end 302b of the splice 302. The suture implant device 300 can include a needle 320 on the fixed loop 304. The suture implant device 300 can include a member 322 on the adjustable loop 314 similarly to FIGS. 1A-1B.

In one aspect, the tensioning strand 318 of the adjustable loop 314 extends from any portion of the adjustable loop splice 302, which can be at the second end 302b or any location between the first end 302a and second end 302b. The tensioning strand 318 of the adjustable loop 314 may optionally have an anchor member 326 that inhibits a free end of the tensioning strand 318 from being pulled through the splice 302.

The implant 300 can be formed by using the braided splice 202 (e.g., 302) and strands 204 (304a) and 206 (304b) to form fixed loop 304 as in FIG. 2. The adjustable loop 314 can be prepared as described for the adjustable loop 114 of FIGS. 1A-1B. With regard to strand 202b of FIG. 2, it is used to form adjustable loop 314 when passed back into the braided splice 202 (e.g., 302).

The suture implant device 300 can include the adjustable loop 314 that has the tensioning strand 318 attached thereto so that pulling the tensioning strand 318 cinches the adjustable loop 314 closed. This allows a practitioner to shorten the adjustable loop 314 of the implant 300. The adjustable loop 314 has a member 322 on it, where the member 322 may be a bone anchor that fastens into the bone. The implant 300 includes a fixed loop 304 that has a needle 320 on it that allows the practitioner to be able to whipstitch a tendon or other tissue in a fashion that is more like a baseball stitch. The needle 320 on the fixed loop 304 allows the practitioner to lock the suture in a way that is useful and to be able to whipstitch the implant 300 on the tendon or other tissue. The fixed loop 304 with the needle 320 on it is connected to the splice 302. The fixed loop 304 extending from the first end 302a of the splice 302 and having the needle 320 on the fixed loop 304 allows for a unique whipstitching.

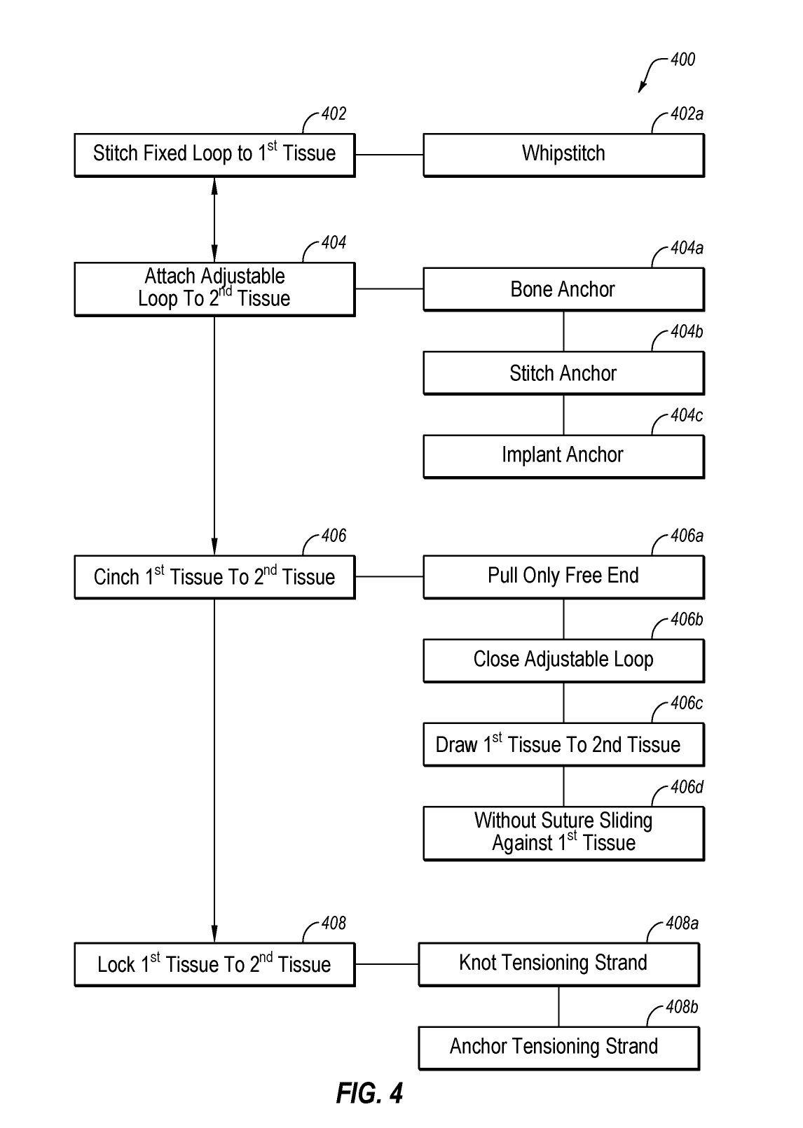

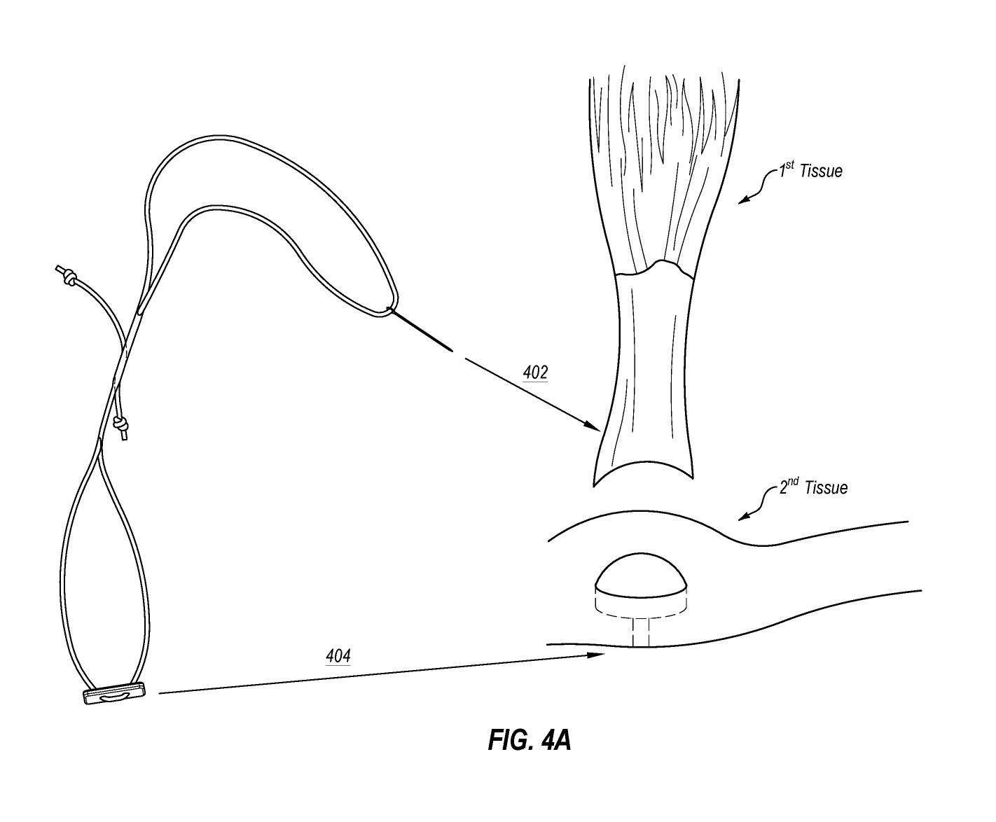

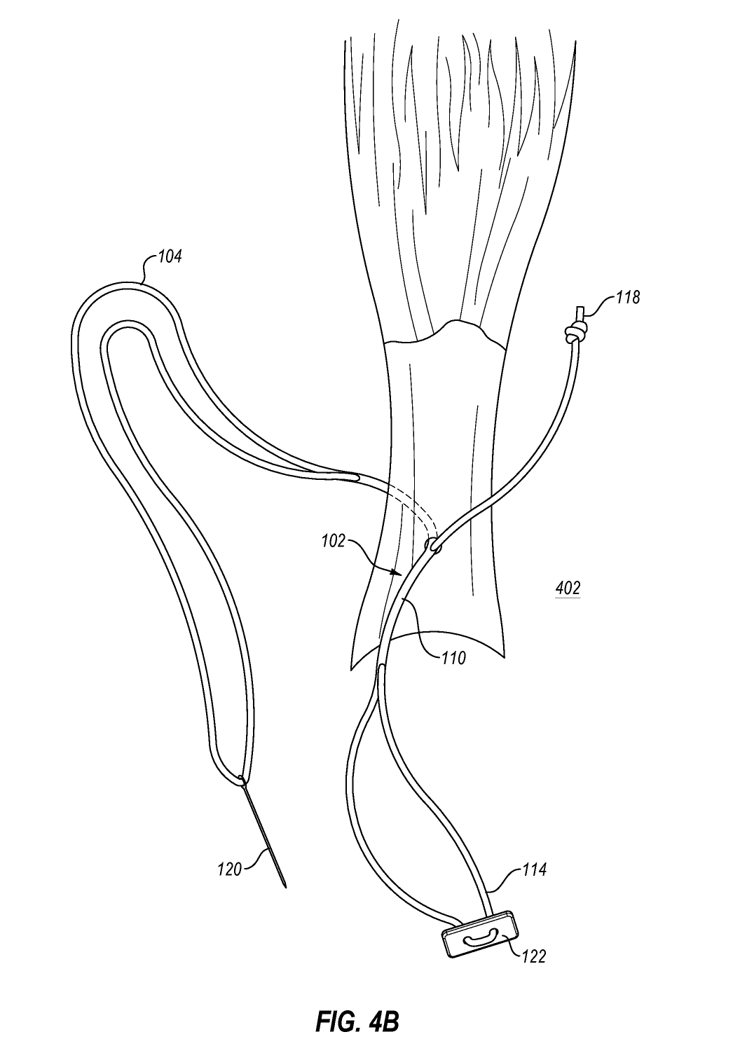

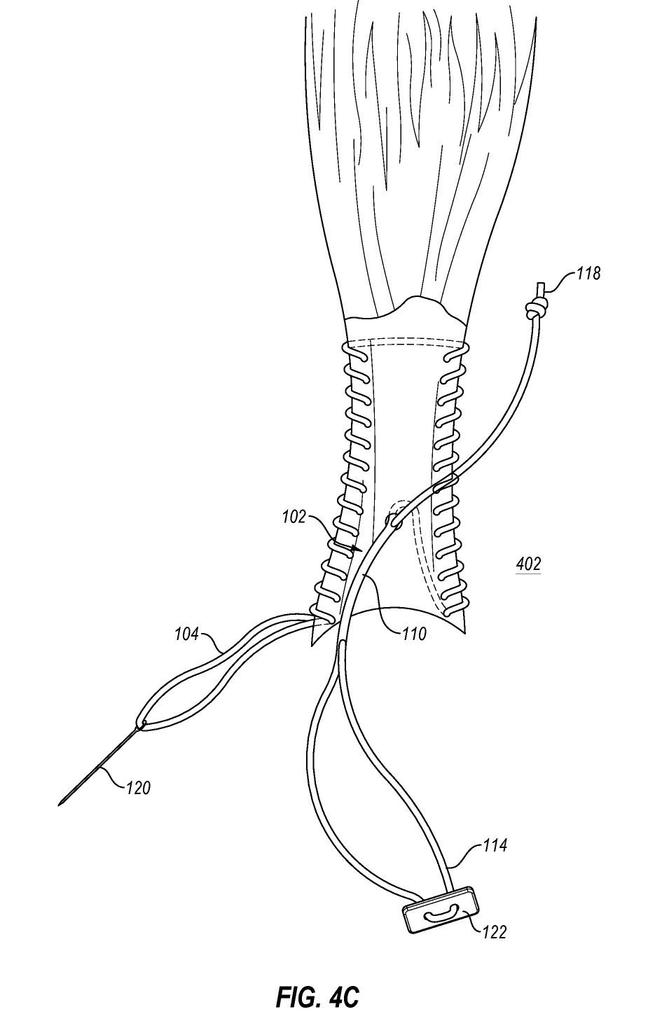

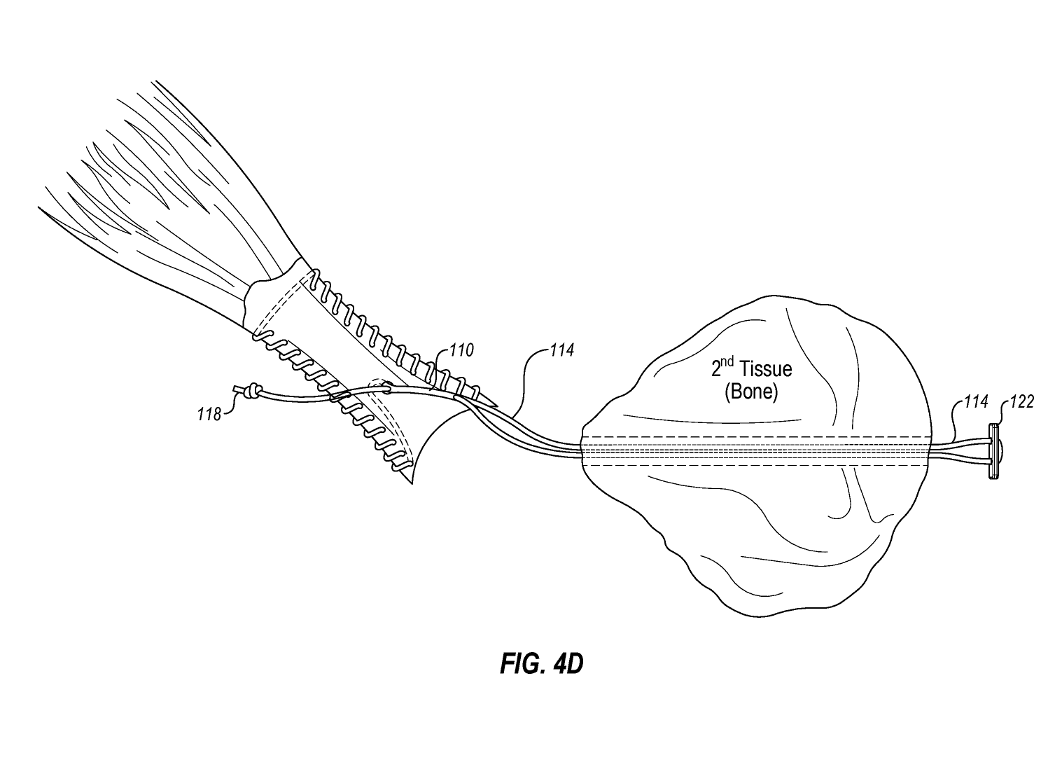

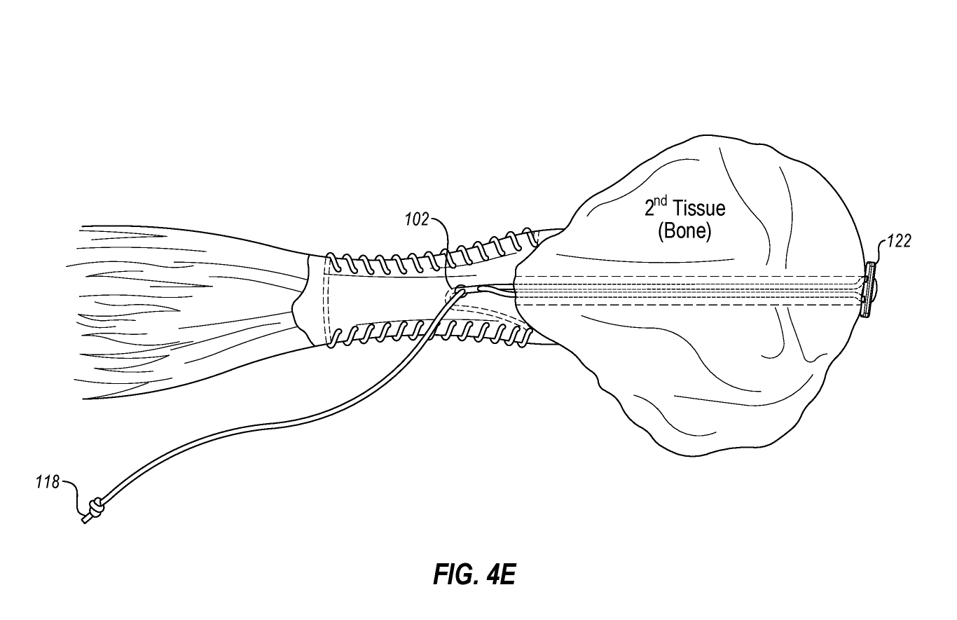

In one embodiment, the suture implant can be a self-cinching suture implant that can be used in a suturing method (400) shown as in FIG. 4 where the suture implant can be stitched to the first tissue (Step 402), such as via a whipstitch or other multi-stitch (Step 402a), and then the member on the adjustable loop is connected to another tissue or another implant (Step 404; bone anchor 404a, stitch anchor 404b, implant anchor 404b, etc) to cinch the first tissue to the other tissue or other implant (Step 406). The suture implant has a fixed loop (e.g., the circumference or length of the loop is fixed from the start point back to the start point of measurement, such as at a sheath) at one end and adjustable loop at the other, where the fixed loop can be stitched into the first tissue (402) more than one time, such as a multi-stitch (e.g., exemplified by being whipstitched into a soft tissue or optionally to bone). Most often, the first tissue can be a soft tissue, such as a tendon or ligament or cartilage or meniscus, but it may be a harder tissue such as bone in other instances. Often, the second tissue is a hard tissue or tissue harder than the first tissue, where suture sliding against the tissue may be allowable without damaging the second tissue. However, the second tissue often receives a bone anchor or other device to fasten to the second tissue, which also results in no sliding of a suture against the second tissue (404).

The end with the fixed loop has a sheath that has a portion of the suture passing therethrough so that the portion of the suture is adjacent to or protrudes from the fixed loop, and the portion of the suture has an opposite end with a knot or member (e.g., slide blocker) that is dimensionally too large to be pulled through or otherwise pass through the sheath. See FIGS. 1A-1B. The suture implant has the adjustable loop placed through a second implant (e.g., button, suture, screw, plate, bone anchor, or any implant of any type, such as any orthopedic device, which can be the member 122 or 322) or second tissue, and then the second implant is then coupled to a second tissue as known in the art, such as by a bone anchor. The second tissue can be prepared to receive the second implant, such as to prepare a hole or receptacle to receive the member on the adjustable loop. The free end is then drawn through the splice so that the cord of the implant extends through the sheath with the free end of the cord being pullable. This will allow the suture implant to cinch the first tissue down to the second implant or second tissue (406). The cinching can be done by pulling the free end (406a) of the tensioning strand to close the adjustable loop (406b), thereby drawing the second tissue to the first tissue (406c). This allows the placement of the second implant or second tissue to be adjacent to the first tissue and first implant. This is performed so that the suture implant is coupled with bone (e.g., second tissue) or in a hole created in the bone to dock a tendon (e.g., first tissue) to the bone. During the cinching, the use of multiple stitches of the fixed loop of the suture implant into the first tissue inhibits the suture implant from sliding against the first tissue (406d). Accordingly, the configuration and use of the suture implant allows the ability to run a stitch through soft tissue or bone (e.g., first tissue) and hook to another implant (e.g., second implant, member 122 or 322), and then to be adjusted and be tensioned to pull the first tissue to the second tissue to lock them together without any part of the suture sliding against either the first tissue or the section tissue when being cinched together. Thus, the inventive suture does not damage the tissue or cause tearing or cutting of the tissue due to the suture sliding against the tissue. This provides significant benefits in terms of healing and overall recovery of the surgical procedure. Once the first tissue and second tissue are satisfactorily coupled together with the implant, the tensioning strand is used to lock the first tissue to the second tissue (Step 408). The locking of the first and second tissue can be by tying a knot into the cinched tensioning strand (408a) or anchoring the tensioning strand (408b) with a member (e.g., clip, tied back into implant, first tissue, tied to whipstitch, tied to multi-stich, anchor etc.).

FIG. 4A shows the implant 100, 1^{st} tissue, and 2^{nd} tissue. FIG. 4B shows the fixed loop 104 being coupled to the 1^{nd} tissue, and FIG. 4C shows the coupling to be a whipstitch. FIG. 4D shows the member on the adjustable loop being inserted through a hole in a bone (2^{nd} tissue). FIG. 4E shows the member being pulled tight against the bone and the tension strand being pulled to cinch the 1^{st} tissue to the 2^{nd} tissue. The adjustable loop and tensioning strand are then fixed so that the adjustable loop is fixed and no longer adjustable. This secures the 1^{st} tissue to the 2^{nd} tissue via the suture implant.

The implants described herein can be used similarly to the other implants of the incorporated references. Here, the needle on the fixed loop can be used to whipstitch or other multi-stitch a first tissue (e.g., tendon). Optionally, the first tissue can be passed through the aperture of the fixed loop (e.g., lassoed), and then the needle can penetrate through the first tissue for multi-stitching. In any event, the fixed loop is fixed to a first tissue and the adjustable loop is fixed to a second tissue via the member on the adjustable loop. The member can be attached to the second tissue. Various configuration of the member, such as bone anchor, can facilitate such attachment. Also, the member on the adjustable loop may be a need to attach the adjustable loop to a soft tissue (e.g., whipstitch, multi-stitch). After the fixed loop is attached to a first tissue and the adjustable loop is attached to a second tissue, the tensioning strand attached to the adjustable loop can be tensioned and pulled so as to pull the adjustable loop closed so as to cinch the first tissue and second tissue together.

The suture implant can be used in various medical procedures where suturing can be used. In some examples, the suture implant can be used to connect a softer tissue to a harder tissue, such as connecting a tendon to bone. In some instances, the suture implant can be used to connect two soft tissues that may or may not be the same, such as connecting a part of a meniscus to another part of a meniscus, or connecting two hard tissues (e.g., connecting two bone pieces together). In any event, the use of the suture implant can be performed as described herein. The examples provided herein are not limiting, and are merely demonstrative of the capabilities of the uses of the suture implant.

Any button or member (e.g., 122 or 322) described herein can be any anchor, plate, screw or other member, such as a member that aids in facilitating attachment to another tissue, such as bone.

In one embodiment, a splice can be a conduit that is formed within a suture cord such that the suture cord can be the conduit as a lumen within the cord body. The conduit serves as a conduit for another suture cord or portion of the same suture cord. The splice may also be a sleeve or sheath or other member coupled or attached to the suture cord that provides a conduit for another suture cord to pass through.

In some embodiments, a splice can be shared between two loops, such as a fixed loop and adjustable loop. Alternatively, a splice may be split into two separate splices, such as a splice for the fixed loop and a splice for the adjustable loop.

The implants described herein can be prepared with a single cord as described. However, multiple cords with or without one or more sheaths can also be used to prepare the implant.

In one embodiment, a method of cinching two tissues together can be performed with the suture implant devices described herein. Both the device from FIGS. 1A-1B and device from FIG. 3 can be used in the same way. The method is now described. Take the fixed loop put the fixed loop around the first tissue (e.g., tendon) and whipstitch from the fixed loop up to the first tissue. In one aspect, the practitioner can whipstitch the first tissue and then stitch up the first tissue one centimeter (or a suitable distance) and then stitch down the first tissue that one centimeter (or same suitable distance to the starting point). This stitching can lock the fixed loop side of the implant into place on the first tissue. Then after the whipstitch is in the first tissue, the practitioner can cut the needle off of the fixed loop to generate two free ends. The two free ends can be connected together again via being tied or an anchor member or clasp clamping the two strands together. The member on the adjustable loop is then connected to the second tissue, whether it is a hard tissue or a soft tissue. The hardness can determine the configuration of the member being a needle or a bond anchor. When a tissue, the member can be a needle and stitch (e.g., whipstitch) the second tissue. When a bone, the member can be a bone anchor that is inserted into the second tissue. In one aspect, the practitioner can drill a hole in the bone to receive the member. Once the implant is attached to the first and second tissues, the tensioning strand can be pulled so as to close the adjustable loop and cinch the first tissue and second tissue together. This can suck the first tissue into the socket of the second tissue.

In one embodiment, a suture implant device can include: a fixed loop splice; a fixed loop having a first fixed end at a first end of the fixed loop splice and the fixed loop having a second fixed end at the first end of the fixed loop splice, wherein optionally one of the first fixed end or second fixed end forms the fixed loop splice; an adjustable loop splice having a first end and a second end that is adjacent to, spaced from, connected with or integrated with a second end of the fixed loop splice; an adjustable loop having a fixed end at the first end of the adjustable loop splice and the adjustable loop having an adjustable end at the first end of the adjustable loop splice, wherein optionally the fixed end forms the adjustable loop splice and the adjustable end passes through the adjustable loop splice so that a tensioning strand of the adjustable loop protrudes from the second end of the adjustable loop splice; a needle on the fixed loop; and a member on the adjustable loop.

In one aspect, the other end of the first fixed end or second fixed end of the fixed loop terminates in the fixed loop splice. In one aspect, the other end of the first fixed end or second fixed end of the fixed loop protrudes from the fixed loop splice. In one aspect, the other end of the first fixed end or second fixed end of the fixed loop protrudes from the fixed loop splice and has an anchor member on its end to inhibit being drawn into the fixed loop splice. In one aspect, the needle is slidably on the fixed loop.

In one aspect, the member is slidably on the adjustable loop. In one aspect, the member is a button, suture, screw, plate, bone anchor, or any implant of any type, such as any orthopedic device.

In one aspect, the member that attaches to the second tissue includes a body, button, anchor, or second implant having one hole or at least two spaced apart holes each being dimensioned to receive the cord of the adjustable loop therethrough.

In one aspect, the adjustable loop splice has a second end that is at least partially integrated with a second end of the fixed loop splice.

In one aspect, the tensioning strand of the adjustable loop extends from any portion of the adjustable loop splice. In one aspect, the tensioning strand of the adjustable loop has an anchor member that inhibits a free end of the tensioning strand from being pulled through the adjustable loop splice.

In one embodiment, an integrated splice can include: a single strand braided splice having a first end and a second end; a first strand braided from a first end of the braided splice and extending therefrom; and a second strand braided from the first end of the braided splice and extending therefrom. In one aspect, at least a portion of the braided splice is a fixed loop splice. In one aspect, the first strand and second strand connect to each other or to the needle to form a fixed loop. In one aspect, the braided splice is a splice for a fixed loop and splice for an adjustable loop. In one aspect, an adjustable loop extends from the second end of the braided splice (e.g., braided splice forms adjustable splice). In one aspect, an anchor member is on the braided splice that inhibits the braided splice from being pulled through a tendon or other tissue.

In one embodiment, a suture implant device can include: a splice having a first end and a second end; a fixed loop having a first fixed end at the first end of the splice and the fixed loop having a second fixed end at the first end of the splice, wherein at least one of the first fixed end or second fixed end forms the splice; an adjustable loop having a fixed end at the second end of the splice and the adjustable loop having an adjustable end at the second end of the splice, wherein the adjustable end passes through the splice so that a tensioning strand of the adjustable loop protrudes from the splice, the fixed end of the adjustable loop optionally being formed from the splice; a needle on the fixed loop; and a member (e.g., anchor or needle) on the adjustable loop. In one aspect, the fixed end of the adjustable loop is of the same cord that forms the splice, optionally being continuous from the splice. In one aspect, the fixed end of the adjustable loop is attached to the splice. In one aspect, the other end of the first fixed end or second fixed end of the fixed loop terminates in the fixed loop splice. In one aspect, the other end of the first fixed end or second fixed end of the fixed loop protrudes from the splice. In one aspect, the other end of the first fixed end or second fixed end of the fixed loop protrudes from the splice and has an anchor member on its end to inhibit being drawn into the splice. In one aspect, the needle is slidably on the fixed loop. In one aspect, the member is slidably on the adjustable loop. In one aspect, the member is a button, suture, screw, plate, bone anchor, or any implant of any type, such as any orthopedic device. In one aspect, the member includes a body, button, anchor, or second implant having one hole or at least two spaced apart holes each being dimensioned to receive the cord of the adjustable loop therethrough. In one aspect, the tensioning strand of the adjustable loop extends from any portion of the splice. In one aspect, the tensioning strand of the adjustable loop extends from the first end of the splice. In one aspect, the tensioning strand of the adjustable loop extends from an intermediate portion of the splice. In one aspect, the tensioning strand of the adjustable loop has an anchor member that inhibits a free end of the tensioning strand from being pulled through the splice.

In one embodiment, a method of cinching two tissues together without the suture sliding against or through a first tissue is provided. Optionally, the method includes the suture not sliding against or through the first tissue and second tissue. The method can include providing the implant of one of the embodiments herein. The fixed loop is stitched to the first tissue, optionally with a whipstitch or other multi-stitch, such as when the implant is pulled against the first tissue for cinching, the suture cord does not slide relative to the first tissue, and thereby the suture cord does not rub against the first tissue during the cinching, which helps protect the first tissue from damage.

In one aspect, the method includes performing a surgical technique that has a tendon not attached to a bone, and where the bone has a hole formed therein. In one aspect, the method includes preparing the tendon and preparing the bone so that the tendon can be attached to the bone. This can include forming the hole in the bone to receive the implant to attach the tendon to the bone by being anchored in the hole.

In one aspect, the method includes the needle forming a hole in the tendon (however, the hole can be pre-formed) so that the needle and cord pass through the tendon. The cord can be passed through the tendon all the way to the sheath and/or fixed loop splice and/or adjustable loop splice, adjustable loop and/or anchor on the fixed loop splice (that is attached to the fixed loop strand), where the anchor can inhibit the rest of the suture from being pulled through the tendon hole. While the tendon hole can be substantially any suitable place, the example places the tendon hole so that the fixed loop is 1 cm above the end of the tendon.

In one aspect, the method includes the needle forming a hole in the tendon (however, the hole can be pre-formed) so that the needle and cord pass through the tendon. The cord can be passed through the tendon so that the splice, adjustable loop and/or anchor are a distance “D” from the tendon hole. While not against the tendon, the anchor on the fixed loop end can inhibit the fixed loop and splice from being pulled through the tendon hole if the cord is pulled too far through the tendon hole. While the tendon hole can be substantially any suitable place, the example places the tendon hole so that the fixed loop is 1 cm-2 cm above the end of the tendon. However, any suitable distance may be fine.

In one aspect, the method includes the needle being used to stitch the tendon with the suture implant so that the cord passes through the tendon multiple times through multiple tendon holes. The total number of tendon holes the cord is stitched through can be 2, 3, 4, or more, where a plurality of stitches are shown. While the stitching is shown to be on both sides of the tendon, such stitching can be on one side or through the middle or any other possible stitching, such as whipstitching. Each stitch can be a locking stitch. The stitching may go up the tendon from the bottom on one side and then back down the other side of the tendon. The implant cord is shown to be started in the middle center of the tendon and then exit out the middle bottom of the tendon; however, it should be recognized that other suitable stitching that forms multiple locking stitches or multiple stitch holes with the cord can be used. Also, the shuttle suture should not be stitched to the tendon. The anchor (e.g., knot) may or may not be against the tendon.

In one aspect, the adjustable loop includes the cord being passed through a button (e.g., or any body or anchor) by being passed through one hole and then passed through a second hole of the button, so that the first pass is in a first direction and the second pass is in a second direction. The button can be attached to a second tissue (e.g., not the tendon, which is the first tissue) where the first tissue will be attached to the second tissue, where the second tissue may often be a bone or some other tissue. The button may also have various configurations, such as a plate having two holes, where one hole may be oblong and/or one can be circular or both oblong or both circular. The button can have an eyelet and a bridge between the holes.

During the cinching there can be tension on the first tissue as well as on the button (or second tissue). During the cinching of the first tissue to the second tissue, the cord does not slip or slide against the tendon or through any hole in the tendon. Thus, the implant provides an improved way of coupling different tissues together without damaging the tissues during the cinching of the tissues together. The first tissue is pulled to the second tissue by the cord of the adjustable loop sliding through the adjustable loop splice by pulling on the tensioning strand of the cord. In one option, once cinched there does not need to be another knot formed on the cord of the tensioning strand, because the suture implant is self-locking upon cinching. A significant benefit is only pulling one cord, such as pulling the free end of the tensioning strand, to have the implant cinch the first tissue to the second tissue so as to self-lock. However, the tensioning strand can be knotted or otherwise fastened back onto the implant so as to lock the adjustable loop in a fixed position so as to no longer be adjustable.

The lack of slippage or sliding of the cord through tendon holes of the first tissue allows for rigid fixation of the tendon (e.g., first tissue) to the bone (e.g., second tissue). This allows the implant to be stationary and fixed to the tendon. Therefore, the suture implant described herein provides a benefit any time that the first tissue is soft tissue, such as a tendon, and it is important for the first tissue to have structural integrity without tearing or ripping during the cinching or any time after completion of the technique. It also inhibits the first tissue from tearing away from the implant.

Also, the configuration allows for only pulling on the free end of the tensioning strand to cinch the first tissue to the second tissue. This allows for easier operation and performance of the technique because of the simple one cord-pulling cinching technique.

In one embodiment, the sheath of the splices (e.g., fixed loop splice or adjustable loop splice or braided splice) can be configured as a finger trap that has the suture cord from either the fixed loop or adjustable loop as the figure in the webbing sheath of the cord being the trap.

In one embodiment, the implant is formed and used so that after the second stitch, and/or after the conclusion of the surgical technique with the suture implant, the cord or any other portion of the implant does not slide or slip against the tendon holes that have the suture implant (or cord) therein. While forming the first hole and subsequent holes, of course the cord needs to slide through the tendon hole; however, after forming a plurality of holes with the cord going through or after stitching the implant to the tendon, the cord does not slide against any of the tendon holes. So, even during the cinching of the first tissue to the second tissue with the implant, the cord of the implant does not slide through the first tendon. Often, the first tissue is a tendon or ligament, so it is beneficial for the cord to not slip against the tendon or ligament and damage it. A lot of tension can even be applied to the tendon with the implant, and still the cord does not slide against the tendon holes by the use of multiple stitches, such as whipstitching. This allows a secure attachment of the implant to the tendon and retention of tendon structural integrity and without damage to the tendon even under high tension such as during cinching.

In one embodiment, a method of cinching a first tissue to a second tissue with the suture implant device of one of the embodiments is provided. The method can include: obtaining the suture implant; attaching the suture implant to a first tissue; attaching the suture implant to a second tissue; and cinching the first tissue to the second tissue with the suture implant without the suture implant sliding against or through the first tissue.

In one embodiment, the method can include: obtaining the suture implant attached to a first tissue and to a second tissue; and cinching the first tissue to the second tissue with the suture implant without the suture implant sliding against or through the first tissue. The method can include forming a hole in the second tissue adapted to receive the suture implant. The method can include forming a hole in the first tissue and passing the fixed loop of the cord therethrough; and forming one or more additional holes in the first tissue and passing the fixed loop of the cord through each of the additional holes. The method can include stitching the first tissue with the fixed loop of the cord so that the cord is drawn taut in the stitches and does not slide relative to the first tissue. The method can include forming a whipstitch into the first tissue. In one aspect, the method can include: forming a hole in the second tissue; passing the body, button, anchor, or second implant having the adjustable loop of the cord through the second tissue hole; and locking the body, button, anchor, or second implant onto the second tissue by the button pressing against the second tissue adjacent to the second tissue hole.

The method of one of the claims can include cinching the first tissue to the second tissue or body, button, anchor, or second implant without the cord sliding through or over the first tissue.

In one embodiment, a method of cinching a first tissue to a second tissue with a suture implant, the method comprising: obtaining a suture implant; attaching the suture implant to a first tissue; attaching the suture implant to a second tissue; and cinching the first tissue to the second tissue with the suture implant without the suture implant sliding against or through the first tissue.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety, including: EP20120188229; EP20120177242; EP20120185616; EP20140159454; EP20130172604; U.S. application Ser. No. 13/102,182; U.S. application Ser. No. 11/541,506; U.S. Application Publication No. US20080082128, U.S. Application Publication No. US20140276992.

Specific reference is also made to the following U.S. Provisional Applications, which are incorporated herein by specific reference in their entirety: 62/180,347 filed Jun. 16, 2015; 62/200,458 filed Aug. 3, 2015; and 62/212,307 filed Aug. 31, 2015.

1. A suture implant device comprising:
a fixed loop splice;
a fixed loop having a first fixed end at a first end of the fixed loop splice and the fixed loop having a second fixed end at the first end of the fixed loop splice;
an adjustable loop splice having a first end and a second end that is adjacent to, spaced from, connected with or integrated with a second end of the fixed loop splice;
an adjustable loop having a fixed end at the first end of the adjustable loop splice and the adjustable loop having an adjustable end at the first end of the adjustable loop splice, wherein the fixed end forms the adjustable loop splice and the adjustable end passes through the adjustable loop splice so that a tensioning strand extending from the adjustable loop protrudes from the second end of the adjustable loop splice;
a needle on the fixed loop; and
a member on the adjustable loop.

2. The suture implant device of claim 1, wherein the fixed end includes a cord extending away from the adjustable loop splice and then back into and through the adjustable loop splice to extend from the adjustable loop splice to form the tensioning strand.

3. The suture implant device of claim 1, wherein the first fixed end and second fixed end include a cord extending away from the fixed loop splice and connect together to form the fixed loop.

4. The suture implant device of claim 1, wherein a cord connected to one of the first fixed end or second fixed end of the fixed loop protrudes from the fixed loop splice and has an anchor member on its end to inhibit being drawn into the fixed loop splice, thereby fixing the fixed loop.

5. The suture implant device of claim 1, wherein the needle is slidably on the fixed loop.

6. The suture implant device of claim 1, wherein the member is slidably on the adjustable loop.

7. The suture implant device of claim 6, wherein the member is a button, suture, screw, plate, bone anchor, or any implant of any type, such as any orthopedic device.

8. The suture implant device of claim 1, the member comprising a body, button, anchor, or second implant having one hole or at least two spaced apart holes each being dimensioned to receive the cord of the adjustable loop therethrough.

9. The suture implant device of claim 1, wherein the adjustable loop splice has the second end that is at least partially integrated with a second end of the fixed loop splice.

10. The suture implant device of claim 1, wherein the tensioning strand of the adjustable loop extends from any portion of the adjustable loop splice.

11. The suture implant device of claim 1, wherein the tensioning strand of the adjustable loop has an anchor member that inhibits a free end of the tensioning strand from being pulled through the adjustable loop splice.

12. A suture implant device comprising:
a braided splice having a first end and a second end;
a first strand braided from a first end of the braided splice and extending therefrom;
a second strand braided from the first end of the braided splice and extending therefrom, wherein the first strand and second strand connect to form a fixed loop; and
a third strand braided from the second end of the braided splice and extending therefrom and looped back thereto and passing into a lumen in the second end of the braided splice to form an adjustable loop and further extending through the lumen and from the braided splice.

13. The suture implant device of claim 12, comprising an anchor member on the braided splice that inhibits the braided splice from being pulled through a tendon or other tissue.

14. The suture implant device of claim 12, comprising an anchor member on the third strand opposite of the adjustable loop that inhibits the end of the third strand from being pulled through the braided splice.

15. The suture implant device of claim 12, comprising an anchor member on the adjustable loop.

16. The suture implant device of claim 12, comprising a needle on the fixed loop.

17. The suture implant device of claim 12, wherein the braided splice, first strand, and third strand are formed from a single braided cord.

18. The suture implant device of claim 17, wherein the braided splice, first strand, second strand and third strand are formed from a single braided cord, where the first strand and second strand branch from the braided splice.

19. A method of cinching a first tissue to a second tissue, the method comprising:
obtaining a suture implant;
attaching the suture implant to a first tissue;
attaching the suture implant to a second tissue; and
cinching the first tissue to the second tissue with the suture implant without the suture implant sliding against or through the first tissue.

20. The method of claim 19, comprising:
providing a suture implant device comprising:
at least one splice;
a fixed loop having a first fixed end at a first end of the at least one splice and the fixed loop having a second fixed end at the first end of the at least one splice;
an adjustable loop having a fixed end at a second end of the splice and the adjustable loop having an adjustable end at the second end of the splice, wherein the adjustable end passes through the at least a portion of the at least one splice so that a tensioning strand extending from the adjustable loop protrudes from the at least one splice;
a needle on the fixed loop; and
a member on the adjustable loop.

21. The method of claim 20, comprising:
stitching the fixed loop to the first tissue;
attaching the member on the adjustable loop to the second tissue; and
cinching the first tissue to the second tissue by pulling on the tensioning strand to cinch close the adjustable loop.

22. The method of claim 21, comprising releasing the tensioning strand so that the splice traps the cord connecting the adjustable end of the adjustable loop with the tensioning strand within the splice so that inner walls of a lumen of the splice apply pressure to the cord.

23. The method of claim 21, comprising fixing the tensioning strand so that the adjustable loop is fixed and no longer adjustable.
